(19) United States
(12) Patent Application Publication
(10) Pub. No.: US 2017/0007418 A1
(43) Pub. Date: Jan. 12, 2017

(54) FACET JOINT PROSTHESIS

(71) Applicants: IGNITE-CONCEPTS GMBH, Langendorf (CH); RIOS MEDICAL AG, Stans (CH)

(72) Inventors: Tom Overes, Langendorf (CH); Paul Pavlov, Nijmwegen (NL)

(73) Assignees: IGNITE-CONCEPTS GMBH, Langendorf (CH); RIOS MEDICAL AG, Stans (CH)

(21) Appl. No.: 15/113,420

(22) PCT Filed: Jan. 23, 2015
(86) PCT No.: PCT/CH2015/000007

§ 371 Date: Jul. 21, 2016

(30) Foreign Application Priority Data
Jan. 26, 2014 (CH) 103/14

(51) Int. Cl. A61F 2/44

(52) U.S. Cl. CPC A61F 2/4405; A61F 2002/30115

(57) ABSTRACT

The present application relates to a facet joint prosthesis comprising a first component having a first disc with a first outer face for engagement of a first bone. The first outer face has an opposed first inner face, which includes a first post connecting said first disc to a tongue. The facet joint prosthesis further comprises a second component having a second disc with a second outer surface for engagement with a second bone. The second outer surface has an opposed second inner face, which comprises a bridge located near the periphery of said second disc. Said bridge connects the second disc with a third disc. The third disc comprises a pocket. Insertion of said tongue into said pocket and rotation of said tongue relative to said pocket results in a locking of said tongue between said second disc and said third disc.

### TECHNICAL FIELD

The invention relates to a facet joint prosthesis with two components having a pre-defined range of motion relative to each other.

### BACKGROUND ART

The human spine includes multiple vertebrae, wherein an intervertebral disc is located between two vertebrae. The intervertebral disc has a cushioning function and provides flexibility to the spinal column and allows for extension and bending of the spinal column. Each vertebra further comprises a posterior structure, the facet-joints, which provide stability between the adjacent vertebrae. In combination with multiple muscle and ligamentous structures, the spine can bend and extend, but has sufficient constraint to prevent excessive motions.

The facet-joint links and stabilizes a first vertebral body in relation to neighbouring vertebral bodies. A vertebral body forms four facet-joints with its two neighbouring vertebral bodies; a superior left and right joint, and an inferior left and right joint, formed by articulating processes, comprising a cartilage joint surface and a joint capsule for stabilisation. The inferior processes of a vertebral body form a joint with the superior processes of a neighbouring vertebral body.

During motion of the spine, for example bending, extending and rotating, the articulating processes translate against each other. Such motion may cause wear and tear of the cartilage surface. Further, degeneration of the articulating processes may be caused by aging, trauma, excessive loading of the spine, or may be a secondary result of intervertebral disc degeneration. The degeneration of the facet joints causes low back pain, which compromises many daily activities. One conventional form of treatment is injection of facet-joints with an anaesthetic and a corticosteroid anti-inflammatory medication. If conventional treatments do not relieve form pain, surgery is the alternative. One option is to fuse the spine: The bones of adjacent vertebrae are made to grow together by complete immobilisation of the spine. For this intervention, the intervertebral disc is removed, replaced by a spacer allowing bone in- and through growth. Additionally, the posterior side of the spine is immobilised by implantation of a pedicle screw system.

A second option, especially when the intervertebral disc is not significantly degenerated, is to replace the articulating surface of the facet joint and implant with an articulating spacer.

An example of such an articulating spacer is the facet Glyder, from the company Zyga Technology. It comprises two separate small PEEK discs that are placed in the facet joint and replace the joint surfaces.

The discs comprise a rougher structure at the side for engagement with the bone for fixation means. Replacement of the articular surfaces by an artificial layer can eliminate the facet pain. Important is to guarantee that the prosthesis will remain at its intended position in the facet joint and that both the articulating surfaces remain in contact during articulation.

For example, AU 2013237744 (Spinal Elements Inc.) discloses implants for augmentation and restoration of vertebral facet joints. Such an implant comprises a first body and a second body, each of said bodies having a first face and a second face. The first faces of both bodies are adapted to articulate relative to each other, while the second faces are adapted to engage an articular surface of a facet joint. Both the first body and the second body are dimensioned to substantially fit within a joint capsule of the facet joint.

US 2011/0022089 A1 (Zyga Technology Inc.) discloses systems for treating patients experiencing facet joint related pain. This system includes a superior resurfacing device and an inferior resurfacing device. The resurfacing devices comprise a resurfacing body having a disc like shape with a first major surface serving as an articulating surface. Once implanted, the first major surfaces of both the superior and the inferior resurfacing device will bear against each other and form a sliding interface between adjacent vertebral bodies.

The implant systems according to the state of the art allow an unrestricted range of motion. Further, these systems either comprise two elements which move freely relative to each other and hence require special instruments for implantation such that they do not disassemble during the implantation procedure or they include multi-part flexible connections which increase the complexity and cost of production of the implant.

### SUMMARY OF THE INVENTION

It is the object of the invention to create a facet joint prosthesis which allows to limit the range of motion between facets of neighbouring vertebrae.

The solution of the invention is specified by the features of claim 1. According to the invention, the facet joint prosthesis comprises a first component having a first disc with a first outer face for engagement of a first facet process bone and an opposed first inner face. The first inner face includes a first post connecting said first disc to first engagement means, said first engagement means being in the form of a substantially flat tongue having a first maximal length and a first maximal width. Further, the facet joint prosthesis comprises a second component having a second disc with a second outer surface for engagement with a second facet process bone and an opposed second inner face. Said second inner face includes at least one bridge located near the periphery of said second disc. The at least one bridge connects the second disc with a third disc comprising a pocket. The pocket has a second maximal length and a second maximal width. Said second maximal length and said second maximal width of said pocket are larger than said first maximal length and said second maximal width of said tongue, respectively. Further, said first maximal length of said tongue is larger than said second maximal width of said pocket. Insertion of said tongue into said pocket and rotation of said tongue relative to said pocket results in a locking of said tongue between the second disc and the third disc.

As the tongue is re-oriented by the rotation, it will be arranged between the second disc and the third disc in such a way that the tongue may no longer pass through the pocket, as the first length of the tongue is larger than the width of said pocket. Therefore, the first component will be locked with the second component. However, the tongue may still translate between the second disc and the third disc, hence allowing movement of said first component relative to said second component. Abutment of said post on an edge of said pocket limits the movement of said first component relative to said second component. Hence, by varying the position, form and/or size of said pocket it is possible to alter the range-of-motion of said first component relative to said second component. Further, as the locking of both components together does not require any instruments, screw, bolts or the like, the facet prosthesis implant according to the present invention is very easy to assemble and implant.

The first, second and third discs preferably have the same shape and dimensions. Said shape or geometry of said discs preferably is circular, oval, oblong rectangular or polygonal. The discs preferably have flat surfaces. However, said first disc and said second disc more preferably comprise a first outer surface and a second outer surface, respectively, said outer surfaces being indented to contact facet process bones. Hence, said outer surfaces preferably comprise structural elements, such as a plurality of protrusions, ribs or the like to enhance friction between these surfaces and the facet process bones.

The thickness of the facet join implant preferably is 3.5 mm or less. Preferably, the components of the facet joint implant are made of stainless steel, a titanium alloy, cobalt chromium steel or of a biocompatible polymer such as polyetheretherketone (PEEK) or polyetherketoneketone (PEKK).

Preferably, said tongue and said pocket have an oval shape. An oval has a major diameter and a minor diameter. The maximal length of said tongue and said pocket corresponds to the major diameter while said maximal width corresponds to said minor diameter. Alternatively, the tongue and the pocket may have other shapes, e.g. such as oblong with rounded edges. However, it is to be noted that said maximal length always has to be bigger than said maximal width, as otherwise no locking of said first component with said second component could take place. Alternatively, said maximal length might also be smaller than said maximal width. Further, said shape must not comprise any angulate corners, but rather only comprise rounded corners such as to allow a smooth movement of said first component relative to said second component.

As understood herein, “a disc” is a structure having two sides with a length and a width which are substantially bigger than the thickness, i.e. the distance between both sides of said disc.

For the purpose of the present application “substantially flat” means that the tongue may only comprise a slight curvature on either of its sides. However, preferably, said tongue has flat sides connected by a rounded edge.

The post preferably has a circular or oval cross-section. Most preferably, said post is arranged near the edge of said tongue on a virtual line running along said maximal width of the tongue. Hence, the post is eccentrically arranged on said tongue.

The tongue itself is preferably arranged eccentrically on said first inner face of the first disc. The tongue is thereby oriented relative to said first disc such that the post is facing towards the centre of the first disc and such that a virtual line running along the first maximal width of the tongue is aligned with a virtual line running along a diameter of said first disc.

The pocket is arranged on said third disc such that when said first disc and said second disc are oriented congruent with one another, said first maximal length of said tongue is parallel to said second maximal width of said pocket. Preferably, the pocket is arranged symmetrically in said third disc, i.e. the centre of said pocket is congruent with the centre of said third disc.

The bridge is preferably arranged on the edge of said second and third discs and forms a material connection between both said discs. The bridge thereby spans an arc of said second and third disc. The length of said arc as well as the width of the bridge is selected such that a firm connection between said second and said third disc is achieved. Alternatively, more than one bridge, such as two, three or more bridges may be arranged between said second and said third discs. Further alternatively, said second and third discs may be connected circumferentially with each other, i.e. the space between said discs is circumferentially enclosed by the bridge.

Said first disc and said second disc each have a first circumferential perimeter and a fourth circumferential perimeter, respectively, as well as a first thickness and a fourth thickness, respectively. Preferably, the ratio of the first circumferential perimeter to said first thickness and/or the ratio of the fourth circumferential perimeter to said fourth thickness is at least 10:1, preferably at least 20:1.

Hence, the first disc and the second disc have a relatively large surface area of said first and second outer faces, respectively, compared to their thickness. This provides for an optimal contact with spinal process bones while retaining a small overall thickness of the facet joint implant. More preferably, said first thickness and said fourth thickness are equal. Further preferably, said first perimeter and said fourth perimeter are also equal.

Preferably, the third disc has the same ratio between its perimeter and its thickness. Most preferably, said third disc has the same perimeter and thickness as said fourth perimeter and said fourth thickness of said second disc.

Preferably, said tongue has a similar shape as said pocket but with smaller dimensions.

This allows for an easy insertion of said tongue into said pocket.

Said bridge preferably has a fifth thickness which defines a space separating said second disc from said third disc, wherein said tongue has a third thickness which is smaller than said fifth thickness.

This allows a free translational and rotational motion of said tongue between said second disc and said third disc. More preferably, the difference between the third thickness and the fifth thickness is small, such that the tongue may not move in a direction which is perpendicular to the second inner face of said second disc. Alternatively, the third thickness may be selected such that some movement of the tongue in the direction perpendicular to said second inner face is possible. This would also allow some angulation of the tongue relative to the second inner face, i.e. the first component and the second component would also be able to carry out a “wobbling” motion relative to each other.

Said post has a second perimeter and said pocket has a fifth perimeter. Preferably, the ratio between said second perimeter and said fifth perimeter is at least 1:3, preferably larger than 1:7. The smaller the ratio between the second perimeter and the fifth perimeter, the smaller is the possible range of motion of said first component relative to said second component. It was found that a minimal ratio of 1:3 is needed such as to re-establish a natural range of motion between both facet process bones.

Preferably, said first component comprises a first protrusion protruding from said first disc and said second component comprises a second protrusion protruding from said second disc, said first protrusion and said second protrusion each comprising at least one bore for receiving a bone fixation element. Provision of protrusions with bores allows anchoring the facet joint prosthesis to the facet process bones outside of the facet joint itself.

Preferably, said first component and/or said second component comprise a cannulation for receiving a K-wire or a guide wire. This facilitates the implantation of the facet joint prosthesis in a facet joint. More preferably, said cannulation is in the form of a channel on the outer face of the first disc and/or of the second disc. Further, in the case where the facet joint prosthesis comprises protrusions with bores, said cannulation extends through the respective protrusion in the form of a through bore.

Said first component and said second component are preferably monolithically formed as one block of material. More preferably, said first component as well as said second component is milled from a block of material each. Hence, only these elements have to be assembled which greatly reduces the complexity of the implantation procedure.

Other advantageous embodiments and combinations of features come out from the detailed description below and the totality of the claims.

### BRIEF DESCRIPTION OF THE DRAWINGS

The drawings used to explain the embodiments show:

FIG. 1 a first embodiment of a facet joint prosthesis according to the present invention in a perspective view;

FIGS. 2a to 2c detailed views of the first component of the first embodiment of the facet joint prosthesis;

FIGS. 3a to 3c detailed views of the second component of the first embodiment of the facet joint prosthesis;

FIGS. 4a to 4h the different steps in assembling the facet joint prosthesis according to the first embodiment;

FIGS. 5a, 5b partial cross-sectional views of the facet joint prosthesis according to the first embodiment;

FIGS. 6a to 6h different positions of the first component relative to the second component;

FIGS. 7a to 7c different views of a second embodiment of a facet joint prosthesis in which said first and said second components include protrusions;

FIG. 8 a third embodiment of a facet joint prosthesis having a cannulation;

FIG. 9 a section of a spine with an implanted facet joint prosthesis.

In the figures, the same components are given the same reference symbols.

### PREFERRED EMBODIMENTS

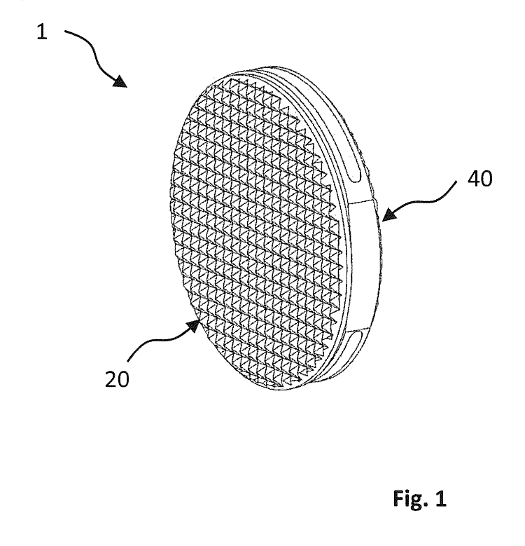

FIG. 1 shows a first embodiment of a facet joint prosthesis 1 according to the present invention in a perspective view. The facet joint prosthesis comprises a first component 20 and a second component 40, wherein said first component 20 and second component 40 are hold together by engagement means which allow for a multi-axial movement of the first component relative to the second component 40 while hindering a disassembly of the first component 20 and of the second component 40.

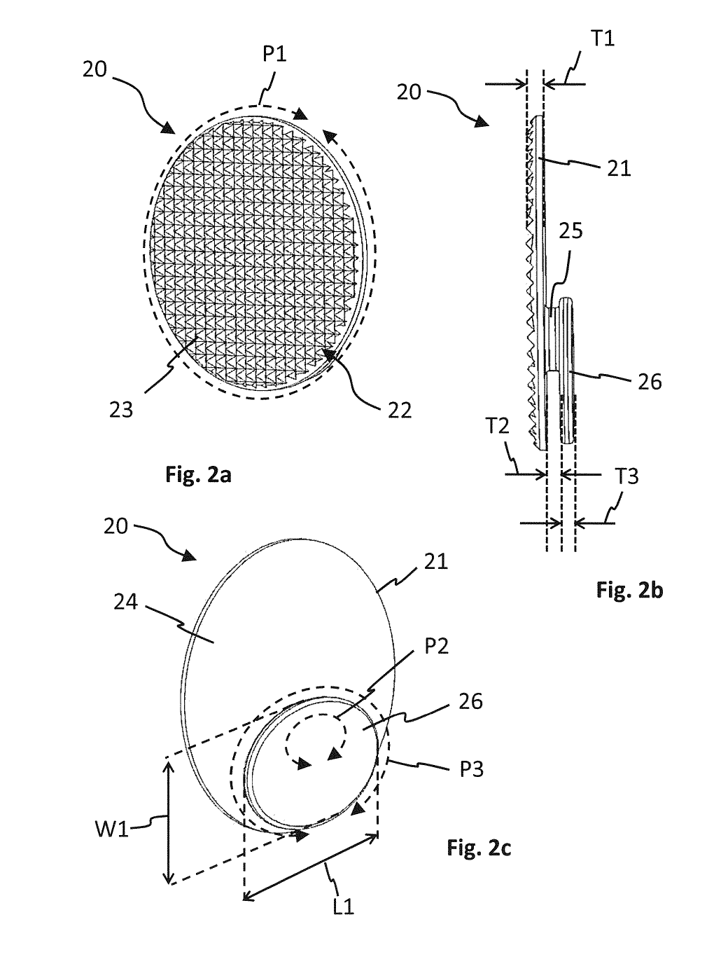

FIGS. 2a to 2c show the first component 20 in greater detail. FIG. 2a is a perspective representation of the first component 20. The first component 20 comprises a first disc 21 with a first outer face 22 for engagement with a first facet process bone. The first disc 21 is preferably circularly shaped, but may be oblong, oval, polygon or irregularly shaped. The first outer surface 22 comprises a plurality of first rough structural elements 23 for bony ingrowth and initial primary fixation. The first disc 21 has a first perimeter P1 and a first thickness T1. The ratio of said first perimeter P1 to said first thickness T1 is at least 20:1.

FIG. 2b shows the first component 20 in a side view. A post 25 connects the first disc 21 to a first engagement means in the form of an oval or oblong shaped tongue 26. The tongue 26 comprises a first maximal length L1 and a first maximal width W1, wherein the first maximal length L1 is larger than the first maximal width W1. As described in greater detail later, the tongue 26 and the post 25 inhibit separation of the first component 20 and the second component 40 and define a pre-defined range-of-motion of the first component 20 relative to the second component 40.

Furthermore, as depicted in FIG. 2c, which represents the first component 20 from the other side of the first disc 21 as FIG. 2a, the first disc 21 comprises a first inner face 24. Said first inner face 24 is connected to the post 25 which extends substantially perpendicular from the first inner face 24 and which has a substantially round, oval or oblong cross-section. The post 25 comprises a second outer perimeter P2 and a second thickness T2.

The tongue 26 has a third perimeter P3 which extends over the second perimeter P2 of the post 25. The tongue 26 is substantially flat, wherein the ratio of the third perimeter P3 to the third thickness T3 of the tongue 26 is at least 10:1.

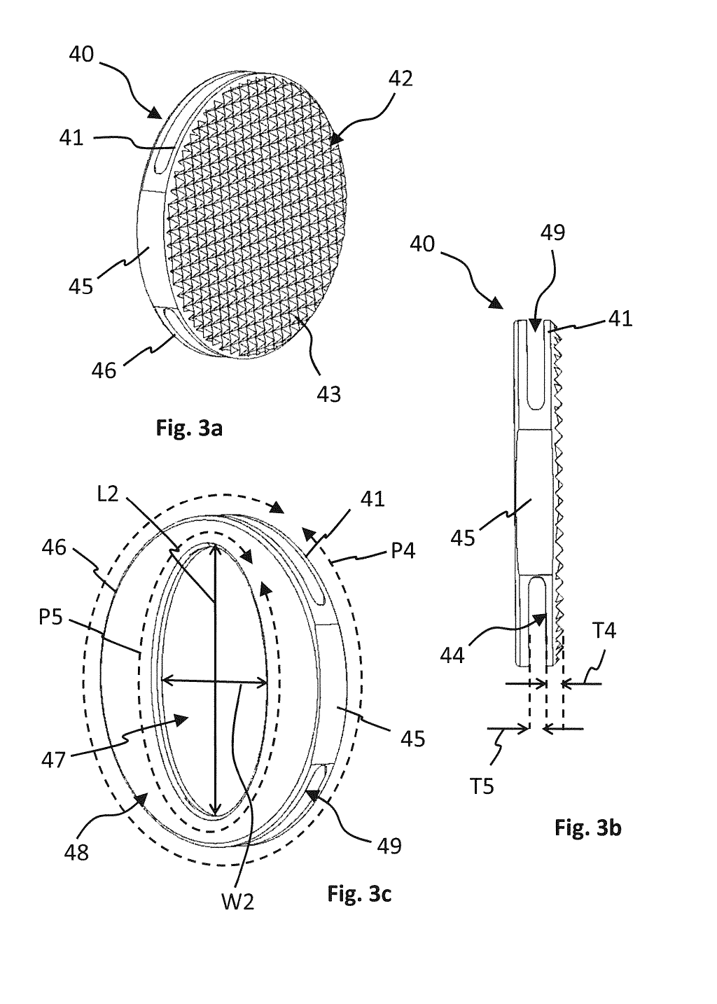

FIGS. 3a to 3c show the second component 40 in greater detail. FIG. 3a is a perspective representation of the second component 40. The second component 40 comprises a second disc 41. The second disc 41 includes a second outer face 42 for engagement with a second facet process bone. The second disc 41 is preferably circularly shaped, but may be oblong, oval, polygon or irregularly shaped. The second disc 41 comprises a second outer surface 42 with second rough structural elements 43 for bony ingrowth and initial primary fixation. The second disc 41 has a fourth perimeter P4 as well as a fourth thickness T4. The ratio of the fourth perimeter P4 to the fourth thickness T4 is at least 10:1.

As shown in FIG. 3b, the second disc 41 comprises a bridge 45 extending substantially perpendicular from a second inner face 44 of said second disc 41, wherein the bridge 45 is located at the periphery of disc 41 and has a fifth thickness T5. The bridge 45 connects the second disc 41 to a third disc 46. The third disc 46 is substantially equal in shape and size as the second disc 41, while the third disc 46 is spaced from said second disc 41 by a space 49.

Furthermore, as shown in FIG. 3c, the third disc 46 comprises an oval or oblong pocket 47. The pocket 47 has a fifth perimeter P5. The pocket 47 comprises a second maximal length L2 and a second maximal width W2, wherein the second maximal length L2 is greater than said second maximal width W2.

The relation between the dimensions of pocket 47 and the tongue 26 of the first component 20 is defined by: L2>L1>W2>W1. I.e. the second maximal length L2 of the pocket 47 is bigger than the first maximal length L1 of the tongue 26, while the second maximal width W2 of said pocket 47 is bigger than the first maximal width W1 but smaller than said first maximal length L1 of said tongue 26.

In a preferred embodiment, the pocket 47 is substantially similar in shape and minimally larger than the tongue 26 of first component 20. Furthermore, the fifth perimeter P5 of the pocket 47 is substantially larger than the second perimeter P2 of the post 25. The third disc 46 furthermore comprises an outer articulation surface 48.

As described in greater detail later, the post 25 and the pocket 47 inhibit the separation of the first component 20 and the second component 40 and define a pre-defined range-of-motion.

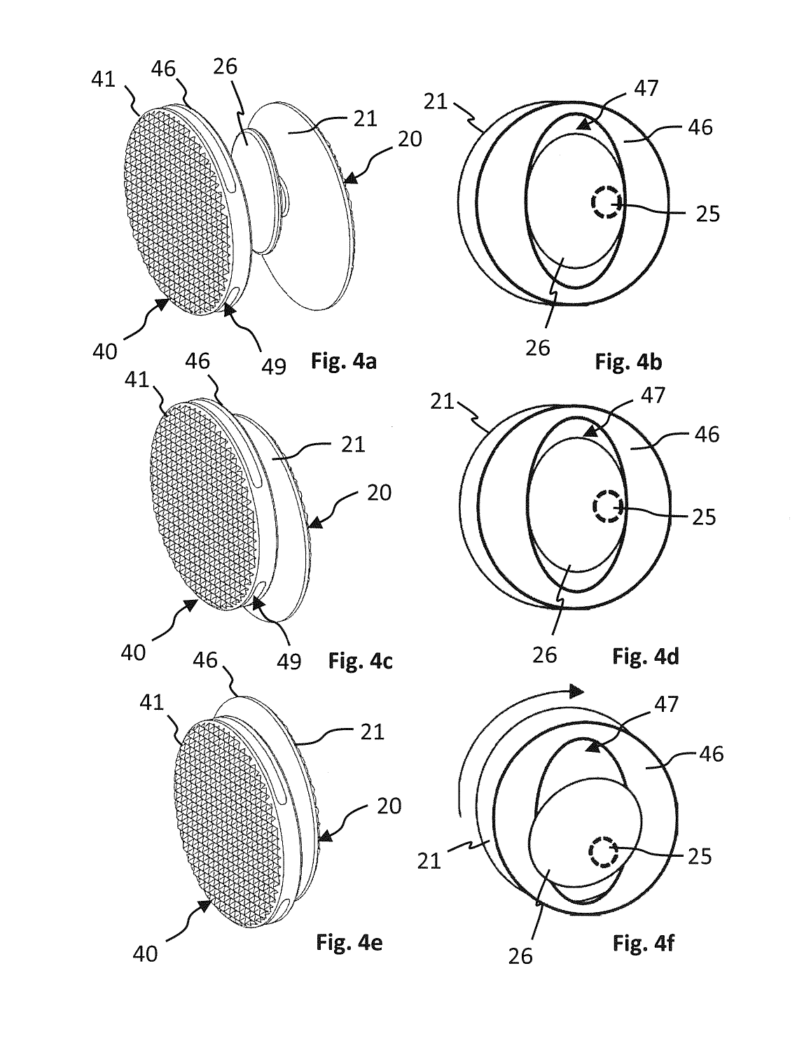

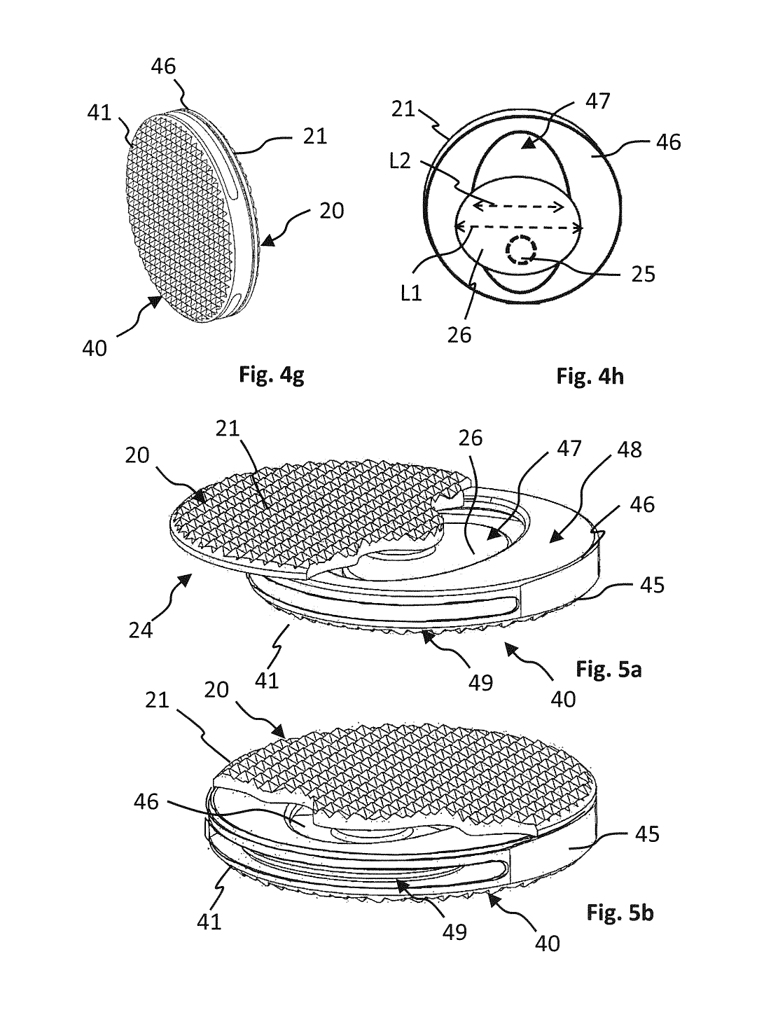

FIGS. 4a to 4h show the different steps in a method of assembly of the first component 20 with the second component 40. The figure on the left shows a perspective view of the assembly step, while the figure on the right of the page shows a schematic representation of the assembly step as viewed from the second disc 41 towards the first disc 21. For illustrative purposes, the second disc 41 is not shown on the right FIGS. 4b, 4d, 4f and 4h.

In the first step as shown in FIGS. 4a and 4b, the tongue 26 of the first component 20 is aligned with the pocket 47 of the second component 40 such that the first maximal width W1 of the tongue 26 lies substantially parallel to the second maximal width W2 of the pocket 27. Note that the location of the post 25 is indicated by dashed lines on said tongue 26.

In a second step, shown in FIGS. 4c and 4d, the tongue 26 is inserted into the pocket 27. As the first maximal width W1 and the first maximal length L1 of the tongue 26 are smaller than the second maximal width W2 and the second maximal length L2, respectively, of the pocket 27, the tongue 26 fits into the pocket 27, such that the tongue finally rests within the space 49 located between the second disc 41 and the third disc 46.

In a third step, shown in FIGS. 4e and 4f, the first component 20 is turned by approximately 90° relative to the second component 40.

FIGS. 4g and 4h show the final situation of the first component 20 and the second component 40 after the three assembly steps. As a result of the turning, the tongue 26 is aligned within the pocket 27 such that the first maximal length L1 of the tongue 26 lies substantially parallel to the second maximal width W2 of the pocket 27. The first maximal length L1 of the tongue 26 being larger than the second maximal width W2, the tongue 26 will protrude over a circumferential edge of the pocket 47 within space 49, thus forming a kind of form-fit connection between the first component 20 and the second component 40. It is to be noted that the third thickness T3 of the tongue 26 is smaller than the fifth thickness T5 of the space 49. Hence, the tongue 26 may freely translate between the second disc 41 and the third disc 46 in two directions lying parallel to said second disc 41 and said third disc 46 and rotate there between. Further, depending on the ratio between said fifth thickness T5 and said third thickness T3, the tongue 26 may have a further translational degree of freedom in a direction perpendicular to said second disc 41 and said third disc 46, also allowing the tongue 26 to “wobble” between the second disc 41 and the third disc 46. Such a wobbling may also be achieved by providing the tongue 26 with a curved outer surface.

FIGS. 5a and 5b show partial cross sectional views of the facet joint prosthesis 1 according to the first embodiment. FIG. 5a shows the configuration of the first component 20 and the second component 40 in the first step of the assembly method. The tongue 26 of the first component 20 is inserted into pocket 47 of the second component 40, wherein the articulation surface 48 of the second component 40 abuts with inner face 24 of the first component 20. In this position, the tongue 26 is situated in the space 49 between the first disc 41 and the third disc 46.

In FIG. 5b, the final configuration after the assembly of the facet joint prosthesis 1 is shown. By turning the first component 20 by approximately 90° relative to the second component 40, the tongue 26 is locked in the space 49 between said first disc 41 and said third disc 46. The tongue 26 is inhibited from being pulled out of the pocket 47 since the first length L1 of the tongue is arranged generally parallel to the second width W2 of the pocket 27, wherein said first length L1 is bigger than said second width W2. The post 25 extends from said tongue 26 through said pocket 47 to said first disc 21. The first component 20 can slide and turn relative to said second component 40, as the tongue 26 is freely translatable within said space 49. However, engagement of the post 25 with the edge of said pocket 47 limits the freedom of motion of the first component 20 relative to said second component 40 to a maximal range-of-motion. The maximal range-of-motion may be varied by adapting the design parameters such as the location, shape and/or the second perimeter P2 values of the post 25 in relation to the shape, size, second length L2, second width W2 and/or fifth perimeter P5 values of the pocket 47.

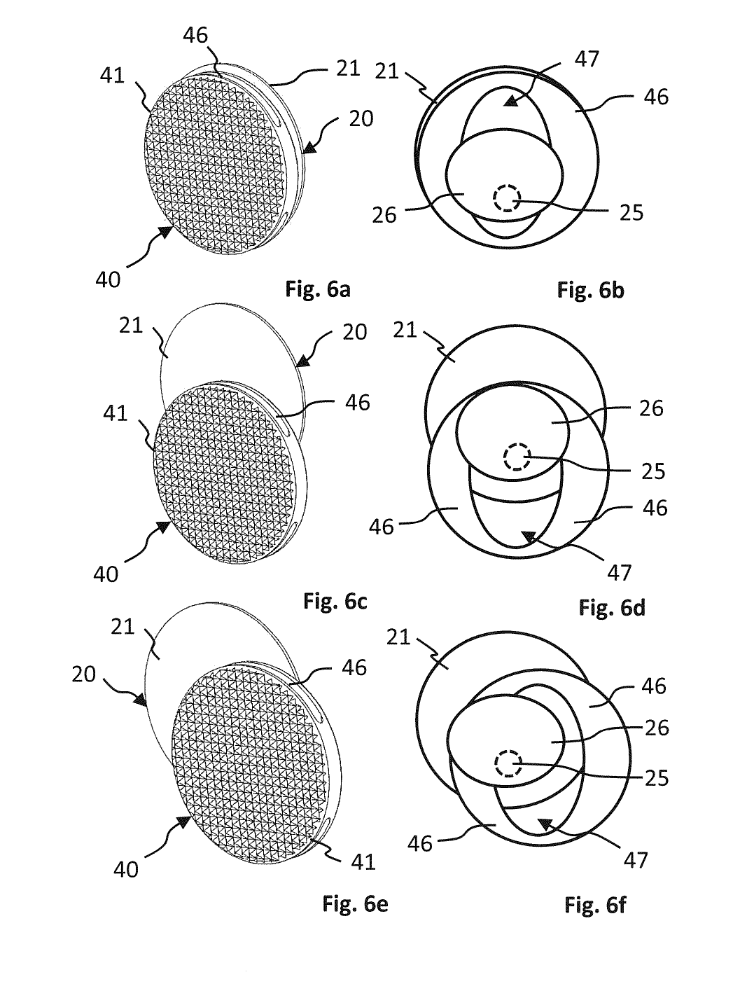

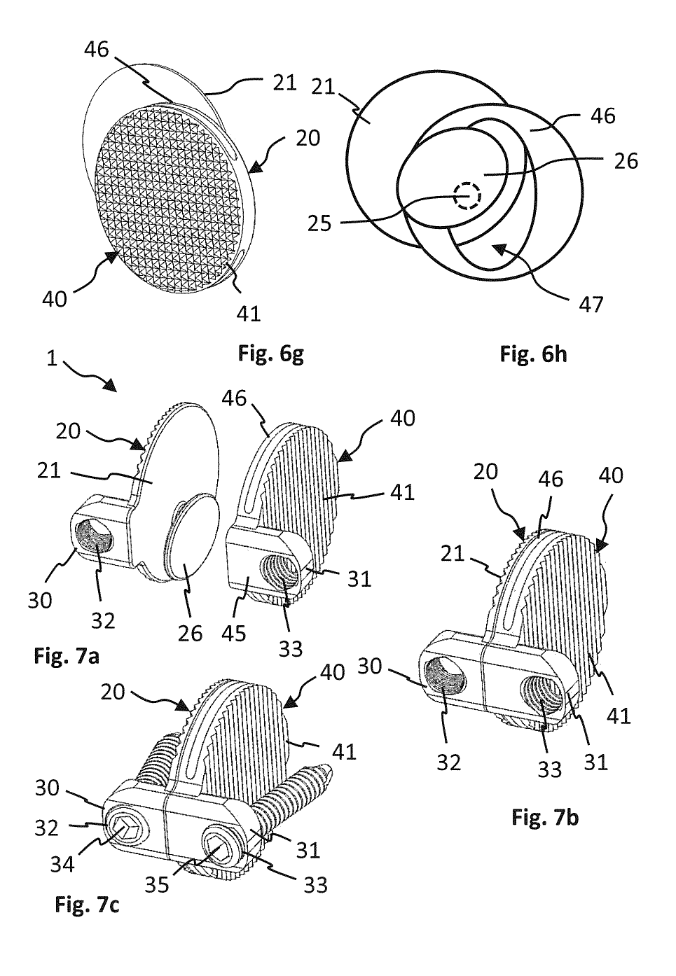

FIGS. 6a to 6h show different positions of the first component 20 relative to the second component 40. As for FIGS. 4a to 4h, the FIGS. 6a, 6c, 6e and 6g on the left side of the page show the facet joint prosthesis 1 in a perspective view, while the FIGS. 6b, 6d, 6f and 6h on the right hand side of the page show a schematic view of the facet joint prosthesis 1 as shown from the second disc 41 towards the first disc 21, wherein the second disc 41 is not shown in said figures for illustrative purposes. The position of the post 25 is indicated by a dashed circle.

FIGS. 6a and 6b show the first component 20 and the second component 40 in an initial position, wherein said first disc 21 and said second disc 41 are arranged essentially congruent with each other.

FIGS. 6c and 6d show a position where the first component 20 is translated along the second length L2 of the pocket 47. In this position, the post 25 abuts on the edge of the pocket 27, hence hindering any further translation of the first component 20 relative to the second component 40.

In FIGS. 6e and 6f, the first component 20 was translated to the left and slightly rotated in relation to the second component 40. In the position as shown in these figures, the post 25 abuts on the edge of the pocket 27 on the left side. It is to be noted that due to the round cross-section of the post 25, the first component 20 may be rotated relative to the second component 40 when abutting on the edge of said pocket 47, e.g. then leading to a position as shown in FIGS. 6g and 6h, where the first component 20 was further rotated—seen in the direction of the observer—in counter-clockwise direction while the post 25 abuts on the edge of the pocket 47.

FIGS. 7a to 7c show a second, alternative embodiment of the facet joint prosthesis 1 according to the present invention. Both said first component 20 and said second component 40 comprise a first protrusion 30 and a second protrusion 31, respectively. Said protrusions 30, 31 protrude from said first disc 21 and said second disc 41. It is to be noted that the second protrusion 31 is arranged in the area of the bridge 45 of the second component 40. The first protrusion 30 is arranged on an edge portion of the first disc 21 which will lie opposite the bridge 45 once the first component 20 and the second component 40 are assembled and said first disc 21, second disc 41 and third disc 46 are all arranged congruently to each other.

The first protrusion 30 and the second protrusion 31 each comprise a first bore 32 and a second bore 33, respectively, for receiving a bone fixation element, such as angle stable screws 34, 35. By usage of a bone fixation element the facet joint prosthesis 1 may be fixated to the facet process. In the embodiment shown, both bores 32, 33 include an internal thread. However, both bores 32, 33 may alternatively be configured without an internal thread.

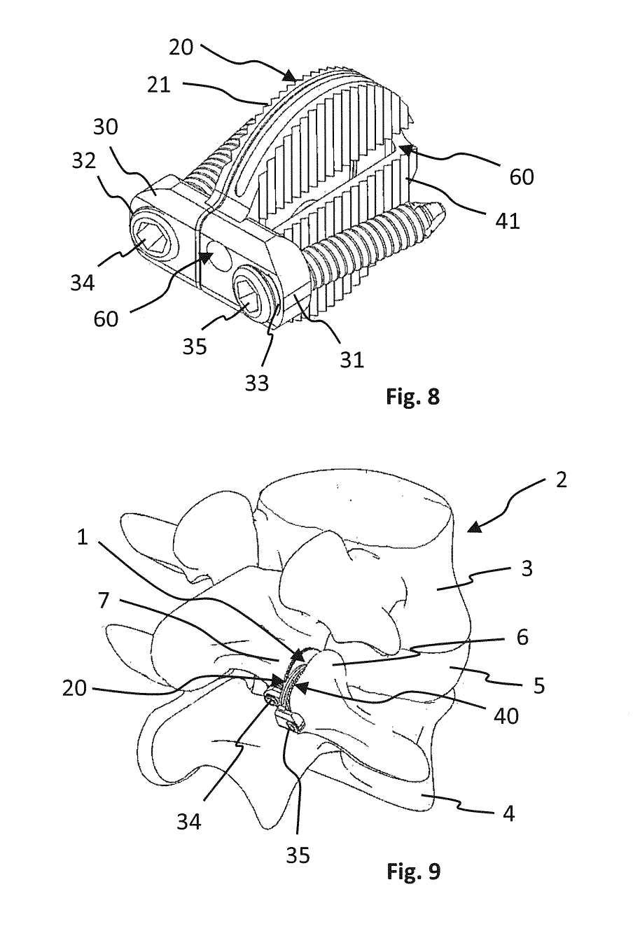

FIG. 8 shows a further embodiment of a facet joint prosthesis 1 according to the present invention. The facet joint prosthesis 1 comprises a cannulation 60 for receiving a K-wire or guide wire arranged on said second component 40. The cannulation 60 spans along the entire second width W2 of said second disc 41 as well as through the second protrusion

31. In the area of the second protrusion 31, the cannulation 60 is in the form of a through bore, while in the area of the second disc 41 the cannulation 60 is in the form of a channel. The cannulation 60 allows the insertion of the facet joint prosthesis over a K-wire or guide wired.

In a preferred surgical technique the facet joint prosthesis 1 in implanted over a guide wire, according to the following surgical steps:

Patient positioning: Prone on the surgical table.

Incision and dilating of soft tissue by usage of stepped dilators to visualise the facet joint.

Insertion of a guide wire into the facet joint.

Preparation of the facet joint by using a rasp for minimal bone removal, wherein the rasp is guided by the guide wire.

Insertion of the facet joint prosthesis 1 over the guide wire.

Insertion of bone fixation elements to fixate the first component 20 and second component 40 of the facet joint prosthesis 1 to the facet process.

FIG. 9 shows a section of a spine 2 with an implanted facet joint prosthesis 1. Shown in this figure are an upper vertebra 3 and a lower vertebra 4. An intervertebral disc 5 is located between both vertebrae 3, 4. The facet joint prosthesis 1 is arranged between a superior articular process 6 of the lower vertebra 4 and in inferior articular process 7 of the upper vertebra 3. As shown, the first component 20 is secured by means of the first angle stable screw 34 to the inferior articular process 7 of the upper vertebra 3, while the second component is secured by means of the second angle stable screw 35 to the superior articular process 6 of the lower vertebra 4.

I claim:

1. A facet joint prosthesis comprising:
a) a first component having a first disc with a first outer face for engagement of a first facet process bone and an opposed first inner face, said first inner face including a first post connecting said first disc to first engagement means, said first engagement means being in the form of a substantially flat tongue having a first maximal length and a first maximal width;
b) a second component having a second disc with a second outer surface for engagement with a second facet process bone and an opposed second inner face, said second inner face including at least one bridge located near the periphery of said second disc, said at least one bridge connecting the second disc with a third disc comprising a pocket, said pocket having a second maximal length and a second maximal width,
wherein said second maximal length and said second maximal width of said pocket are larger than said first maximal length and said first maximal width of said tongue, respectively, said first maximal length of said tongue being larger than said second maximal width of said pocket, wherein insertion of said tongue into said pocket and rotation of said tongue relative to said pocket results in a locking of said tongue between said second disc and said third disc, and wherein the bridge only partially peripherally encloses the space between said second and third discs.

2. The facet joint prosthesis according to claim 1, wherein said first disc and said second disc each have a first circumferential perimeter and a fourth circumferential perimeter, respectively, as well as a first thickness and a fourth thickness, respectively, wherein the ratio of the first circumferential perimeter to said first thickness and/or the ratio of the fourth circumferential perimeter to said fourth thickness is at least 10:1, preferably at least 20:1.

3. The facet joint prosthesis according to claim 1, wherein said tongue has a similar shape as said pocket with smaller dimensions.

4. The facet joint prosthesis according to claim 1, wherein said bridge has a fifth thickness which defines a space separating said second disc from said third disc, wherein said tongue has a third thickness which is smaller than said fifth thickness (T5).

5. The facet joint prosthesis according to claim 1, wherein said post has a second perimeter and said pocket has a fifth perimeter, wherein the ratio between said second perimeter and said fifth perimeter is at least 1:3, preferably larger than 1:7.

6. The facet joint prosthesis according to claim 1, wherein said first component comprises a first protrusion protruding from said first disc (21) and said second component comprises a second protrusion (31) protruding from said second disc, said first protrusion (30) and said second protrusion (31) each comprising at least one bore (31, 32) for receiving a bone fixation element.

7. The facet joint prosthesis according to any of claim 1, wherein said first component and/or said second component comprises a cannulation for receiving a K-wire or a guide wire.

8. The facet joint prosthesis according to claim 1, wherein said first component and said second component are monolithically formed as one block of material.

9. The facet joint prosthesis according to claim 1, wherein said tongue is eccentrically arranged on the first inner face of said first disc.

10. The facet joint prosthesis according to claim 1, wherein said first post is eccentrically arranged on said tongue.

11. The facet joint prosthesis according to claim 1, wherein said second and third discs are both flat.
